(19) United States
(12) Patent Application Publication
(10) Pub. No.: US 2008/0221426 A1
(43) Pub. Date: Sep. 11, 2008

(54) Methods and apparatus for detecting misapplied optical sensors

(75) Inventors: Clark R. Baker, Newman, CA (US); Brian Earp, Castro Valley, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Pleasanton, CA (US)

(21) Appl. No.: 11/716,384

(22) Filed: Mar. 9, 2007

(51) Int. Cl. A61B 5/00

(52) U.S. Cl. 600/407; 600/310

(57) ABSTRACT

Methods and apparatus are described for sensing misplacement of an optical sensor on a patient. A wavelength of light which is particularly subject to absorption by a physiological characteristic of interest is used to compare to a reference to determine if the sensor placement is appropriate, such as to generate accurate readings. In one example, the intensity of a wavelength of light which is subject to absorption by bulk tissue, but less subject to absorption by oxygen in the blood will be detected to evaluate the placement of the sensor.

### TECHNICAL FIELD

The present disclosure relates generally to methods and apparatus for operating optical sensors for sensing physiological characteristics of a patient, and more specifically relates to methods and apparatus for determining that such optical sensors, such as for example, pulse oximetry sensors, are misapplied on a patient.

### BACKGROUND

The use of optical sensors to evaluate one or more physiological characteristics of a patient is well known. One such use, pulse oximetry, is used to determine the level of oxygen saturation in a patient's blood. Many configurations of sensors are known or contemplated for oximetry sensors. In one common type of sensor, the sensor will include two emitters of optical radiation, such as LED's, configured to generate optical radiation, such as visible and near-visible light, of different wavelengths absorbed differently by transmission through the blood and tissue of a patient's body. In many typical configurations, such oximetry sensors include one emitter of optical radiation in a red wavelength, and another emitter of optical radiation in a near-infrared (IR) wavelength. Such sensors will also include a photodetector capable of detecting the energy emitted by the LED's. Variations of oximetry sensors have also been proposed where a third or even more wavelengths of optical radiation would be used to determine additional physiological conditions of the patient.

Oximetry sensors are constructed in different forms to enable attachment to different portions of a patient's body. Because of the requirements of different placements on locations of the body, oximetry sensors operate on at least two different measurement principles. One type of oximetry sensor, of a configuration such as might be placed on a fingertip, transmits the energy directly through the tissue site, in this example through the finger, to the detector. Such sensors are known as transmission sensors. The other basic configuration of oximetry sensor allows the emitters and detector to be arranged on the same surface, such that the detector will receive optical radiation transmitted into the tissue and reflected back to the emitter. Such sensors are known as reflectance sensors, and may be used where the optical radiation cannot transmit through the portion of the body where the sensor is to be placed. One application requiring use of a reflectance sensor is, for example, on a patient's forehead.

Sensors are calibrated relative to their intended usage. Thus, sensors will be designed and calibrated depending on whether their intended use is as a transmission or reflectance sensor, and will be calibrated for a specific spacing, or range of spacings, between the emitters and the detector. Thus, even two transmission sensors, such as one intended for use on a fingertip and another intended for use on an earlobe, will typically have different calibrations. The calibration differences between a transmission sensor and a reflectance sensor are typically greater.

Sometimes, caregivers will misapply an oximetry sensor. Notwithstanding instructions and illustrations on sensor packaging, caregivers may fail to appreciate the differences between sensors and their applications, and may apply a sensor to the wrong portion of a patient's body. For example, a bandage-type transmission sensor intended for use on a fingertip, and which would normally be folded over or around the fingertip, may be unfolded and applied to another portion of the patient's body in a configuration like a reflectance sensor. However, in such a circumstance, not only are the placement of the sensor and measurement method different from what was intended, but the spacing between the emitters and detector is significantly different from what was intended for the sensor. Thus, the misapplied sensor will not give accurate readings for the patient. Other misapplications of a sensor include placement on a site which, although positionally correct, is not suitable for optimal measurements. This situation may exist, for example, when the physical characteristics of the site are unsatisfactory to yield reliable measurements.

Accordingly, there is a need for a system to inform a caregiver when an optical sensor such as an oximetry sensor is misapplied, so that erroneous readings of a patient's physiological condition are not observed and relied upon.

### BRIEF DESCRIPTION OF THE DRAWINGS

The drawings referenced herein depict examples of systems which may implement the present invention. As will be readily apparent to those skilled in the art, these examples are illustrative only, and many other systems and methods may be used to implement and benefit from the present invention.

FIG. 1 is a schematic diagram illustrating a pulse oximeter system according to one example of the invention.

FIGS. 2A-2D are views illustrating examples of different optical sensor configurations and placements.

FIG. 3 is a flowchart illustrating a method of sensing misapplication of an optical sensor according to various embodiments of the invention.

### DETAILED DESCRIPTION

The present invention will be described in the context of detecting misapplication of an optical oximetry sensor, such as may be used to determine a patient's oxygen saturation, and sometimes pulse rate. However, an oximetry system is only one example of an optical sensor with which the present invention may be used, and the invention may be used in optical sensors where additional or different physiological conditions are be determined through measurement of optical radiation engaging a tissue site. For example, optical sensors may be used to measure other physiological parameters, such as, for example, blood glucose, water saturation, CO₂ content, perfusion, etc.

As noted above, an optical pulse oximetry system will use one or more emitters of optical radiation to provide optical radiation at a plurality of different wavelengths. Although the principles of pulse oximetry are well known to those skilled in the art, briefly, the measurements are based on the property that hemoglobin and bulk body tissue absorb optical radiation of different wavelengths at different rates. The various constituents in the hemoglobin and the bulk tissue have different optical properties, including different absorption coefficients. Thus, the amount of optical radiation absorbed by the hemoglobin and body tissue at each wavelength is proportional to the product of the concentrations and absorption coefficients of the respective constituents that are present in each, relative to that wavelength, and to the length of the paths traversed by the optical radiation at that wavelength. For example, bulk body tissue, such as soft tissue, includes water, proteins and fats. Hemoglobin takes several forms, each with its own set of wavelength-dependent absorption coefficients, including deoxyhemoglobin, oxyhemoglobin, carboxyhemoglobin, and methemoglobin. Pulse oximetry measures variations in detected optical radiation between multiple parts of the cardiac cycle, such as systole and diastole, and estimates oxygen saturation as an empirically calibrated function of the ratio of these variations for the red and near-infrared wavelengths. This pulse-based method minimizes the influence of bulk tissue constituents, or of non-pulsing venous blood, on the oxygen saturation estimate.

Because of the different levels of absorption of wavelengths by different tissue and constituent compositions, an indication of sensor misplacement can be obtained from the detected intensity of optical radiation of a selected wavelength after traversing such tissue. Accordingly, use of an oximetry sensor having at least three different wavelengths can enable determination of both oxygen saturation, (denoted as SpO₂), and improper or undesirable sensor placement on the patient.

As noted previously, a typical optical pulse oximetry system will use optical radiation in a red wavelength and a near-IR wavelength. The red wavelength will typically be within a range of about 620 nm to about 760 nm, and the near-IR wavelength will typically be within a range of about 820 nm to about 970 nm. In many preferred examples of oximetry systems, the red wavelength will be within the range of 640 to 690 nm, and the near-IR wavelength will be within the range of 870 to 940 nm. In certain examples of systems, a wavelength of about 660 nm. is preferred for the red wavelength, and a wavelength of about 890 nm. is preferred for the near-IR wavelength. These examples will be used in descriptions herein.

Such a pulse oximeter may be designed to operate in either a transmission or a reflectance mode, either mode operating on the principle that optical radiation that is neither transmitted nor reflected is absorbed. These two wavelengths of optical radiation are selected based on the absorption properties of the oxyhemoglobin, the deoxyhemoglobin and the bulk body tissue. For example, the selected example wavelength in the red band, at about 660 nm, is more strongly absorbed by the deoxyhemoglobin than the oxyhemoglobin, while the example wavelength in the near-IR range, at about 890 nm, is more strongly absorbed by the oxyhemoglobin than the deoxyhemoglobin. The relative amounts of optical radiation absorbed at about 660 nm and 890 nm wavelengths may be compared through algorithms known in the art to determine an SpO₂ concentration.

One or more additional wavelengths may be selected which are relatively unaffected by either deoxyhemoglobin or oxyhemoglobin, and which are more strongly absorbed by the bulk tissue constituents such as water, proteins and fats. When such a wavelength is introduced into a patient and detected, absorption is primarily due to the bulk tissue through which the optical radiation passes. Thus a measurement of the signal intensity for light transmitted at such wavelength may be used as an indicator of the length of the path traversed through the soft tissue.

One example of a wavelength for making such a determination is optical radiation in the range of approximately 1150 to about 1350 nm., with a preferred range being between approximately 1200 to approximately 1300 nm. An example using a wavelength of approximately 1200 nm. will be described herein. Because detection of received optical radiation at such a wavelength is indicative of the distance traversed by the radiation through the tissue, it can be used to determine whether the sensor has been applied to the wrong bulk tissue location. Additionally, in some cases, the absorption may be used to evaluate the properties of the tissue traversed. Because absorption is based on the constituents of the bulk tissue, body area areas having significant differences in composition, for example the bridge of the nose as contrasted with the sole of the foot, may be used in the determination through correlation to appropriate reference values.

One example of a method for making such a determination is to functionally relate the detected radiation intensity, for example at 1200 nm, to a reference to evaluate whether the detected intensity is consistent with an appropriate application of the sensor on the patient. Such functional relation may be a simple comparison or ratio (typically a ratio computed from the logarithms of the intensity signals), or may be a more complex evaluation to the one or more reference value or values. Such reference value may include one or more stored values, for example, indicative of, or functionally related to, a value or range of values of an expected optical radiation intensity of that wavelength for that sensor, if properly applied; or of a measure of absorption of the emitted wavelength for a sensor appropriately placed on a tissue site of the type the sensor was designed to evaluate. Such values may be determined theoretically or empirically, such as through examination of a sample of patients sufficient to yield reliable data. Alternatively, the reference may be either the detected optical radiation intensities at either of the wavelengths used for the oximetry measurement, or a signal or measurement derived from such detected intensities.

As one example of this latter implementation, because the absorption of optical radiation at about 1200 nm, for example, is more strongly absorbed by the bulk tissue than at the wavelengths typically used for oximetry, the detected optical radiation intensity for a transmission mode sensor incorrectly used on the patient as a reflection mode sensor can be substantially less than expected. This is due to the transmission path through the bulk tissue being substantially longer than intended, resulting in greater absorption of the radiation. Furthermore, the bulk tissue absorption coefficients at 1200 nm are substantially higher than at the wavelengths typically used for oximetry measurements. Therefore, a comparison of the detected optical radiation intensity at 1200 nm with one or both of the primary oximetry wavelengths may be used as a measure of the sensor placement. Because such a relationship of the longer wavelength to one or more of the oximetry wavelengths is patient-specific, it may be easier to use such a comparison to yield measurements indicating acceptable placement of a sensor, than to use a single-wavelength reference to one or more pre-determined reference values which will be based on a patient population, rather than the specific patient.

Where the reference includes one or more stored values, the values may be stored in the sensor monitor for access during use. It is known in the art for sensors to include identifying information, including calibration information, sensor type, etc., which is read by a monitor to enable proper control of the sensor and processing of the data from the sensor. The reading of such sensor information by a monitor will allow the monitor to reference one or more values expected when that sensor is appropriately applied to a tissue site.

Alternatively, the reference value or values may be stored in the sensor. For example, when the conventional sensor-identifying information mentioned above is programmed into the sensor, the placement-identifying reference values may be similarly programmed into a flash memory or other appropriate storage in the sensor assembly. Such values may then be accessed by the monitor to make the determinations as described. In some implementations of the present invention, it will be preferred to have reference values of the intensity of the light emitted from the sensor, at least at the wavelength used as the reference for transmission through the bulk tissue (1200 nm. in the present example). For example, the intensity may be determined at the time of emitter and/or sensor manufacture by measuring the amount of light received from the emitter while it illuminates an intensity standard, such as a white surface, at a fixed distance. That intensity information, or other information derived from or functionally representative of that measured intensity information will then preferably be programmed or otherwise stored in or associated with the sensor, in the same manner as is other sensor information, as identified above. Such information indicative of the emitted intensity will facilitate calibration with either stored reference values or another detected intensity signal. Where the reference value will be another detected intensity, it is preferred that there be information indicative of the intensities of the emitters for each wavelength employed in the reference, or at least of some correlation between the intensities of the wavelengths, to facilitate the relative calibration or normalization of signals during a patient evaluation process. Such calibration or normalization will facilitate improved accuracy in any ratios or other comparisons used in evaluation the sensor placement.

In some examples of the invention, it may be desirable to evaluate the application of the sensor relative to a consideration other than correct position on the patient. In such cases, the sensor may include an emitter of optical radiation of a wavelength which is particularly absorbed by an additional patient characteristic pertinent to that other consideration. For example, an oximetry sensor should typically be applied to a patient in an area in which the blood is subject to pulsatile circulation, and is homogenously distributed within the tissue bed, and thus is indicative of the true oxygen saturation of the patient. If a sensor is applied to an area, for example, where there is venous pooling, where the blood is accumulating, or over a large artery, the measured oxygen saturation will typically not accurately correspond to the true oxygen saturation of the patient's circulating blood. Thus, applying an oximetry sensor in an area of venous pooling, or over a large artery, even if the application is positionally correct, may lead to inaccurate readings of the patient's condition.

To address such a situation, a wavelength of optical radiation which is particularly subject to absorption by blood will be selected. For example, an emitter of optical radiation in the green band, such as in the range from about 490 nm to about 590 nm can be used in the sensor. For the current discussion, a wavelength of about 510 nm, will be used as one suitable example. A wavelength within the identified broad range is selected because the absorption of optical radiation by the hemoglobin and oxyhemoglobin at a wavelength within that range is significantly greater than at either of the red or near-IR wavelength bands otherwise used for the oximetry measurement. Therefore, in a manner similar to that described above, the differences in the detected optical radiation intensities in this green range may be compared to one or more reference values to identify if the sensor has been misapplied to a region were venous pooling exists, or where an artery is present, and is thus likely to result in erroneous readings of the a physiological characteristic of the patient, such as oxygen saturation.

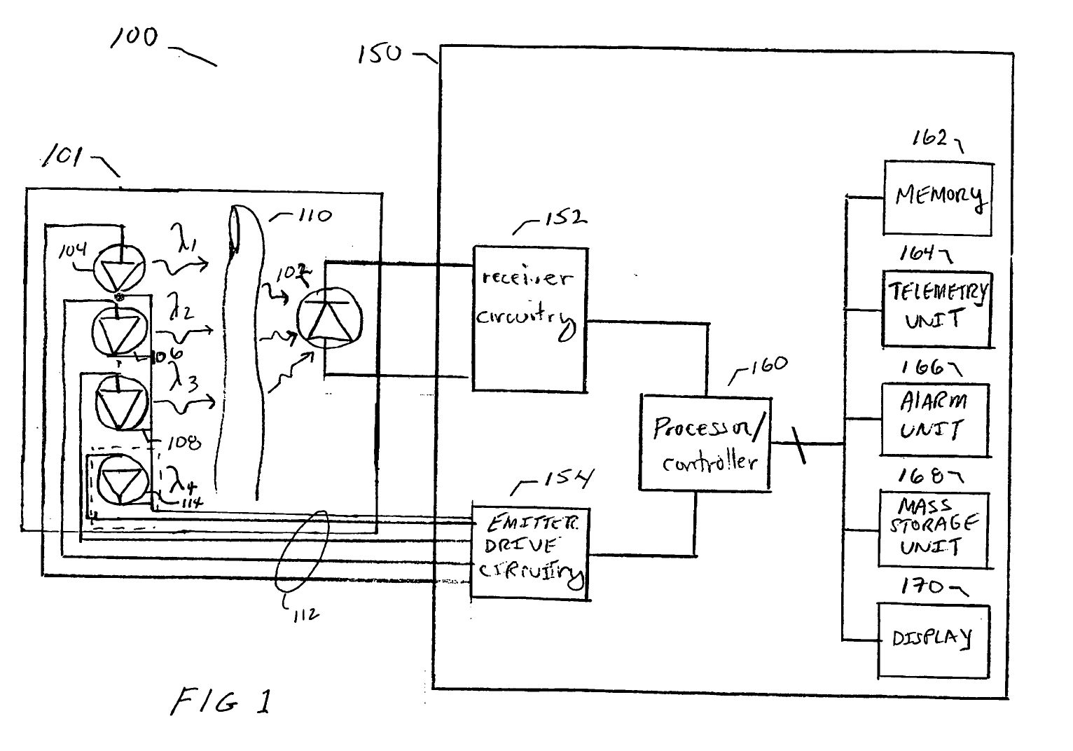

FIG. 1 is a schematic diagram illustrating one example of a pulse oximetry system 100 according to various embodiments of the invention. In this example, the pulse oximetry system 100 includes an optical sensor 101 and a monitor 150. The optical sensor 101 is shown configured to emit at least three wavelengths of optical radiation, λ₁, λ₂, and λ₃ transmitting through bulk human tissue 110. Here, optical sensor 101 includes a photodetector 102 and a three emitters 104, 106, 108 operating at different wavelengths, λ₁, λ₂, λ₃, respectively, as described earlier herein. In this example, the emitters will emit optical radiation at approximately 660 nm, 890 nm and 1200 nm. However, these wavelengths are examples only, and the use of other wavelengths within the ranges identified above, and even beyond those ranges is contemplated. The optical sensor 100 can include more or fewer emitters, depending on the desired area of tissue application. For example, the sensor could include a fourth emitter operating in the green band, as discussed above. Accordingly, an optional fourth emitter 114 (depicted in a dashed line box), is depicted. Alternatively, the emitter 108 operating at approximately 1200 nm., might be replaced with an emitter operating in the green band. And alternatively, additional emitters at other wavelengths may be added, such as to provide reference signals for the radiation intensities as discussed above. As yet another alternative, some optically-based measurements of other physiological parameters may include use of a wavelength which is sensitive to the composition of the bulk tissue, but is relatively insensitive to O₂ in the blood. In such systems, no emitters beyond those also used for the parameter measurement may be required.

The emitters may be of any suitable type known in the art. In many implementations, each emitter will be a light emitting diode (LED). Further, as is known in the art, multiple emitters may be formed in a single package. The photodetector 102 may be of any suitable type, material, or combination of materials known in the art, such as, by way of example only, an avalanche photodiode, a PN junction diode or a PIN diode. It is also envisioned that in some examples, the photodetector 102 and/or the emitters 102, 104, 108 may be located in the monitor 150 and coupled to optical sensor 101 and then to the tissue 110 using one or more optical fibers. Alternatively, the optical radiation sources could be created from one or more sources of a broader spectrum of optical radiation appropriately filtered to provide each desired wavelength.

As noted above, the optical sensor 101 can include a memory 110 for storing information associated with the optical sensor, such as a sensor identifier, a tissue identifier, and one or more baseline or reference values. Some examples of baseline values that may be stored in a sensor memory include, information corresponding to, at one or more wavelengths, the amplitude, phase, or shape of the pulse for an intended tissue location. The memory can further include calibration data related to operation of the emitters 102, 104, 108, such as bias voltages and bias currents, as is known in the art.

An exemplary monitor 150 is schematically depicted in FIG. 1. Monitor 150 may be either a “stand alone” monitor, either stationary or portable, or may be an assembly configured for inclusion in a patient multi-parameter monitoring system. Monitor 150 includes receiver circuitry 152 and emitter drive circuitry 154 coupled to a processor/controller 160. The emitter drive circuitry 154 is further coupled to the emitters 104, 106, 108 by a cable assembly 112. The emitter drive circuitry 154 can include voltage sources, current sources and the like, and a switching fabric as is known in the art to selectively turn on and off the emitters 104, 106, 108 according to signals received from the processor/controller 160. The receiver circuitry 152 is also coupled to photodetector 152 through cable assembly 112, to receive signals associated with the optical radiation sensed by the photodetector 102. The receiver circuitry 152 will typically include signal processing circuitry, such as a digital signal processor and an analog-to-digital converter. The processor/controller 160 accepts the information from the receiver circuitry 152 for further processing and/or storage. Mechanisms other than cable connections have been proposed in the art for establishing suitable connection between the monitor 150 and the optical sensor 101. For example, wireless telemetry systems have been proposed for establishing such connection, and may be used to establish the needed connections in some implementations of the invention.

In various examples of systems, the monitor 150 includes a display 170 to display one or more parameters regarding the patient or monitor operation. For example, the monitor 150 may display a determined oxygen saturation value and/or waveform, a pulse rate, an indicator of the signal intensity and the like. The monitor 150 includes a memory 162, such as volatile and non-volatile memory as is known in the art, and may include a mass storage unit 168, such as a magnetic hard drive and/or removable disk device. The memory 162 and the mass storage device 168, either alone or in combination, can store instructions and executable code for operating the monitor and sensor, and for analyzing the optical radiation signals sensed by the photodetector 102, as described herein. The memory 162 and the mass storage device 168 can also be used to store data transmitted by the receiver circuitry 152 for further processing and transmission.

Additionally, in accordance with the present invention, the monitor 150 may include an indicator of a misapplied sensor. Such an indicator may be a visible indicator, an audible alarm, or both. In various examples of monitors, and as depicted in FIG. 1, the monitor 150 includes an alarm unit 166, such as an audio or visual alarm unit that operates in combination with the processor to provide an alert that a monitored parameter has gone outside of an expected or acceptable range. Monitor 150 may utilize alarm unit 166 to provide an indication of sensor misplacement, as described herein. The monitor 150 can further include a telemetry unit to transmit alarm-related information to a clinician or to a remote central location, such as a nurse's station in a hospital or nursing home environment.

It should be understood that the above description of a pulse oximetry system 100 is intended to provide a general understanding of possible pulse oximetry systems, and is not a complete description of all the elements and features of a specific type of pulse oximeter, as such is well within the knowledge of persons skilled in the art. Further, as noted earlier herein, many examples of the invention are equally applicable to any size and type optical sensor; and the description of pulse oximetry sensors and a pulse oximetry system is merely an example of one system to which the present invention may be applied.

Referring again to FIG. 1, the example sensor module 101 is a transmission sensor, which detects light transmitted directly through a portion of a patient's body, such as a finger, as depicted. Thus, sensor assembly 101 is adapted to fit the patient so that the optical radiation emitted from emitters 104, 106, 108 at λ₁ λ₂, and λ₃, respectively, is coupled to the tissue 110 containing hemoglobin, oxyhemoglobin where it can be absorbed, such that optical radiation that is not absorbed by the tissue 110 is coupled into the photodetector 102 where it is converted to a photocurrent that is transmitted to the receiver circuitry 152. The ratio of the intensity of the optical radiation received by the photodetector 102 to the optical radiation transmitted by the emitters 104, 106, 108, at each respective wavelength, is a logarithmic expression of the optical radiation absorbed by the constituents in the patient tissue. As such, the intensity of the optical radiation at each wavelength traveling through patient tissue is expected to decrease with increasing tissue optical paths according to the Beer-Lambert law. In the described example where optical sensor 101 includes emitters 102, 104, 106 emitting at wavelengths of about 660 nm, 890 nm and 1200 nm, the optical radiation at about 1200 nm is more strongly absorbed by the human tissue 110 than by the hemoglobin and oxyhemoglobin. Therefore, unexpected changes in absorption of the longer-wavelength optical radiation can be an indicator of unexpected and undesirable separation between the emitters 104, 106, 108 and the photodetector 102 and/or the nature of the tissue traversed. Accordingly, a measure of the sensed optical radiation intensity at about 1200 nm can be used to yield an indicator of sensor misplacement. Also, as discussed previously, that indicator may be determined through comparison to one or more stored reference values; or to another monitored characteristic, such as the measured intensity of optical radiation at another wavelength, such as the intensity at about 660 nm and/or 890 nm in the present example

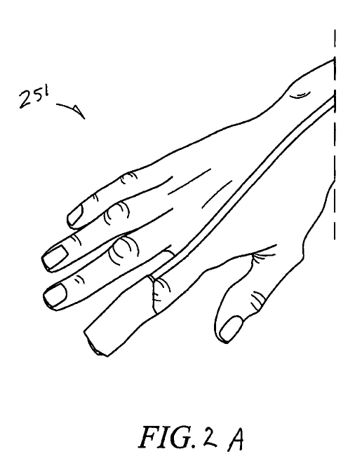

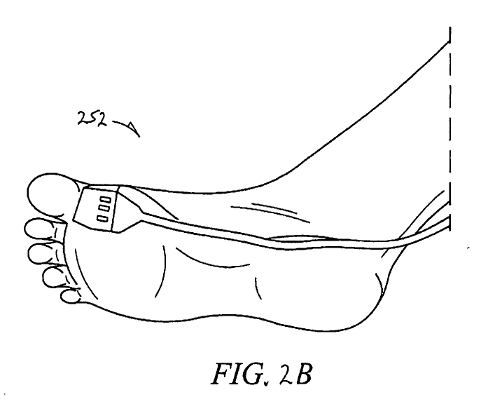

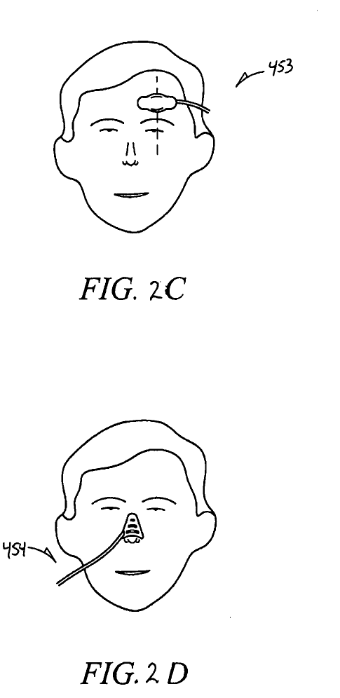

Referring now to FIGS. 2A-D, therein are depicted examples of alternative sensor placements on a patient. The examples of each of these sensors are provided solely to illustrate differences in sensor configurations and placements, and not to illustrate specific sensor constructions. It should be understood that such sensors will include circuits and other structures not addressed herein, but well-known to those skilled in the art.

As noted previously, sensors have been made, and can be envisioned, for attachment to a variety of portions of a patient's body. FIG. 2A depicts one example of a fingertip sensor 251, which may be configured as depicted schematically in FIG. 1 for optical sensor assembly 101, wherein the LED's or other optical radiation sources are placed generally on some side of the finger, while the detector, or detectors, are placed generally on the opposite side, and where the light measured is transmitted through the tissue and blood within the finger. FIG. 2B depicts a foot sensor 252 placed on the sole of a patient's foot. Sensors are also known which may be placed on a patient's toe. In the illustrated examples, both sensors 251 and 252 are bandage-type transmission mode sensors

FIG. 2C depicts a reflectance mode sensor in place on a patient's forehead. With sensor 253, the measured optical radiation is not that transmitted completely through the tissue at the measurement site, but is the light which is diffusely reflected back to the surface at the detector location. Sensors such as sensor 253 may be held in place by a variety of mechanisms, including adhesive bandages, adhesive portions of the sensor, or straps, band or similar devices applying a securing force. FIG. 2D depicts a sensor for placement in the area of the nose 253. Sensor 253 may, in selected examples, be either a transmission mode or reflectance mode sensor. The depicted examples of FIGS. 2A-D are merely examples of an even broader range of sensors.

Given the broad range of sensor configurations adapted for specific placement on a patient, there are a broad range of possible errors in placement that may occur. For example, an transmission mode optical sensor incorporated into bandage material intended for application to digit tissue, such as a finger or toe, may be able to be opened up and misapplied to the forehead. In this situation, the optical sensor, to the extent it may function at all, will function in reflection mode. However, as the calibrations for the sensor will be for use in transmission mode, with a different spacing between the emitters and detector, data obtained using the sensor is almost certain to be erroneous. Regardless of whether this sensor is applied in a transmission or reflection mode, the optical radiation at about 1200 nm will be more strongly attenuated than the optical radiation at about 660 or 890 nm due to substantially higher absorption coefficients of bulk tissue constituents. When this sensor is misapplied in reflectance mode, the optical path between the emitter and detector will be substantially greater than when it is correctly applied in transmission mode. The longer path will result in increased absorption at 1200 nm compared to the shorted path when the sensor is correctly applied in transmission mode. The longer path will also result in an increased absorption difference between 1200 nm and the typical oximetry wavelengths of about 660 or 890 nm. The reduction in the sensed intensity ratio at 1200 nm alone and/or the reduction in a normalized ratio of the sensed intensity at 1200 nm to the sensed intensity, for example, at 890 nm can be used to trigger a signal warning of a possible misapplication. Similarly, the differences in the sensed intensities can be used to provide the indication of sensor misapplication. Because changes in oxygen saturation have a greater influence on optical absorption at 660 nm than at 890 nm, comparisons between 1200 nm and 890 nm absorbance should yield more specific detection of sensor misplacement than comparisons between 1200 nm and 660 nm absorbance.

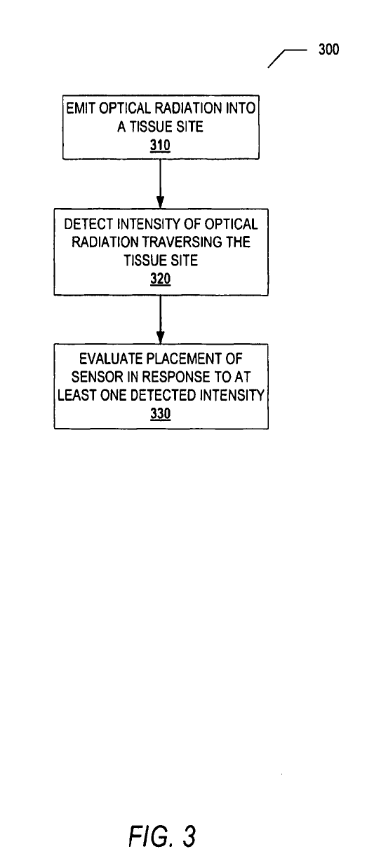

FIG. 3 is a flowchart illustrating a method 300 of sensing misapplication of an optical sensor, e.g. a patient sensor, as may be used in performing some examples of the invention. The method begins at block 310 by emitting optical radiation into a tissue site. At least three wavelengths of optical radiation are emitted, as described earlier herein. Again, more than three wavelengths may be used, for example, to have both a bulk tissue measurement to determine possible application of a sensor to an incorrect body site, and a measurement sensitive to venous pooling to evaluate misapplication to a site which is undesirable, even if positionally correct. At block 320, the emitted optical radiation is detected by photodetector after traversing the tissue site to the detector. In many examples, the detected intensities are transmitted to the monitor 150 for processing of the measured optical radiation.

At block 330 the monitor 150 uses the measured intensity of at least one wavelength to evaluate the placement of the sensor. The monitor will use logic to perform this evaluation. This logic may be in the form of hardware or firmware, but in most cases will be executed in software. As noted previously, this evaluation may be performed either by comparison to one or more stored reference values, or by comparison to another sensed parameter, such as another detected intensity of optical radiation, or a signal derived from such another detected intensity. In the event that the valuation results in a determination of misapplication of a sensor, a notification and/or a record of the determination will be generated.

In many implementations of the invention, each of the above steps, as well as additional operations used to perform each of the above steps, and steps implementing any of the operations as described herein, will be performed by or under the control of one or more processors in the monitor. In such a case, most if not all, of the individual operations required to perform these steps will be implemented in software. In such a case, machine-readable instructions will be contained in, or stored on, a machine readable medium, such as a memory or mass storage device. This machine-readable medium will be in operable communication with the processor, such that the processor may execute the machine-readable instructions, resulting in the performing of the necessary operations to perform the described methods.

Many modifications and variations may be made in the examples of techniques, structures and methods described and illustrated herein without departing from the spirit or scope of the present invention. For example, the wavelengths described herein are illustrative only, and other wavelengths may be used as a measure of the optical path through the bulk tissue, or of another parameter useful in evaluating sensor placement. Additionally, in addition to localized regions of venous pooling or the presence of an artery, there may be other physiological conditions associated with a sensor placement site, that may be evaluated through the basic techniques and methods described herein. Accordingly, the scope of the present invention shall be determined only by the scope of the following claims, and all equivalents of such claims.

What is claimed is:

1. A method of evaluating the application of an optical sensor on a patient, comprising the acts of:
introducing at least one wavelength of optical radiation to the patient, said wavelength selected to be attenuated primarily by bulk tissue of the patient;
detecting said wavelength of optical radiation after attenuation by the tissue of the patient;
comparing the detected optical radiation to a reference; and
in response to the comparison, determining if the optical sensor is misapplied on the patient.

2. The method of claim 1, further comprising the acts of:
introducing at least second and third wavelengths of optical radiation to the patient;
detecting said second and third wavelengths of optical radiation after passing through tissue of the patient: and
determining at least one physiological parameter of the patient in reference to at least said detected second and third wavelengths.

3. The method of claim 1, wherein said at least one wavelength is within the range of 1150 to 1350 nm.

4. The method of claim 3, wherein said second wavelength is within the range of red wavelengths and wherein said third wavelength is within the range of near-infra-red wavelengths.

5. The method of claim 4, wherein said second wavelength is within the range of approximately 620 to 760 nm.

6. The method of claim 4, wherein said third wavelength is within the range of approximately 820 to 970 nm.

7. A method of operating an oximetry sensor having at least three optical radiation emitters, each emitting optical radiation at a wavelength different from the wavelengths from the other emitters, said emitters placed to transmit optical radiation into the tissue of a patient, comprising the acts of:
detecting said three wavelengths of optical radiation after each has passed through the tissue of the patient; and
based on the detection of at least one of said three wavelengths, determining if the sensor is applied appropriately to the patient, and in the event the sensor is not appropriately applied to the patient, generating an indication of the determination that the sensor is inappropriately applied.

8. The method of claim 7, wherein the act of determining if the sensor is applied appropriately to the patient comprises the acts of:
detecting the intensity of light emitted at a first wavelength: and
comparing said detected intensity to a reference to determine if the sensor is appropriately applied to the patient.

9. The method of claim 8, wherein the first wavelength is within the range of 1150 to 1350 nm.

10. The method of claim 8, wherein the first wavelength is within the range of 490 to 590 nm.

11. A method of measuring a physical parameter of a patient through optical radiation, comprising the acts of:
projecting at least three wavelengths of optical radiation into the body of the patient through use of an emitter assembly;
detecting the intensity of each of said three wavelengths of optical radiation after passing through the body of the patient through use of a detector assembly;
determining said physical parameter of the patient in response to the detected intensity of at least a first of said wavelengths; and
evaluating the placement of the sensor in response to the detected intensity of at least a second of said wavelengths.

12. The method of measuring a physical parameter of a patient of claim 1l, wherein said three wavelengths are each with a separate one of the ranges of 620 to 760 nm, 820 to 970 nm. and 1150 to 1350 nm.

13. The method of measuring a physical parameter of a patient of claim 11, wherein said second wavelength is within the range of 1150 to 1350 nm, and wherein said act of evaluating the placement of the sensor in response to the detected intensity of at least one of said wavelengths comprises correlating the detected intensity of said second wavelength to a reference value associated with an expected intensity for an appropriate placed sensor.

14. The method of measuring a physical parameter of a patient of claim 13, wherein the second wavelength is one where absorption of the wavelength is relatively less affected by hemoglobin in a patient's blood than are the first and third wavelengths.

15. An assembly for determining at least one physiological characteristic of a patient, comprising:
a sensor assembly including at least three emitters of optical radiation at different wavelengths, and including at least one detector to detect each of the wavelengths, the sensor configured such that when the sensor is appropriately applied to a patient, the detector assembly detects the intensity of optical radiation of said three wavelengths after said optical radiation has traversed the body of the patient at a measurement site;
a monitor configured for selective attachment to said sensor assembly and to receive signals derived from the detection of said at least three wavelengths of optical radiation detected by said detector, the monitor comprising logic which determines a physiological characteristic of the patient from at least one detected wavelength, the monitor further comprising logic which compares a signal derived from at least one detected wavelength of optical radiation with a reference to evaluate the placement of the sensor on the patient relative to the intended placement of the sensor on the patient.

16. The assembly of claim 15, wherein the logic is implemented at least in part in software.

17. The assembly of claim 15, wherein the reference comprises a stored value.

18. The assembly of claim 15, wherein the reference comprises another detected intensity of optical radiation.

19. The assembly of claim 15, wherein the reference comprises a value derived from another detected intensity of optical radiation.

20. A system for optical sensing of a physiological characteristic, comprising:
an optical sensor comprising a plurality of emitters of optical radiation, the optical sensor configured for attachment to human tissue; and
a monitor coupled to the optical sensor, the monitor comprising:
a processor; and
a machine-readable medium comprising machine-readable instructions, that when executed by the processor, perform operations comprising:
receiving data from the optical sensor, the data associated with detected optical radiation emitted from the plurality of emitters;
analyzing the data received from the optical sensor;
based on the analysis, identifying an inappropriately applied optical sensor; and
generating a signal indicating sensor misplacement if the optical sensor is inappropriately applied.

21. The system of claim 20, wherein at least one of the plurality of emitters is configured to provide optical radiation having a wavelength within the range of either from about 490 nm to about 590 nm, or from about 1150 nm to about 1350 nm.

22. The system of claim 20, wherein the monitor further comprises a memory unit configured to store values used in said analyzing operation.

23. The system of claim 20, wherein the analyzing operation comprises comparing the detected optical radiation intensities at two or more wavelengths.

24. A system for optical sensing of a physiological characteristic of a patient, comprising:
an optical sensor comprising two emitters of optical radiation, the optical sensor configured for attachment to human tissue, where a first emitter emits light within a first near-infrared spectrum, and wherein the second emitter emits optical radiation within the range of 1150 to 1350 nm; and
a monitor coupled to the optical sensor, the monitor comprising:
a processor; and
a machine-readable medium comprising machine-readable instructions, that when executed by the processor, perform operations comprising:
receiving data from the optical sensor, the data including detected intensities of light within each range of wavelengths;
functionally relating the intensity of light detected within the wavelength range of 1150 to 1350 nm to a reference;
based on the functional relation, determining in the optical sensor is appropriately applied to the patient.

25. The system of claim 24, wherein the instructions further comprise instructions which when executed result in the operation of generating an indicator if the optical sensor is determined to be inappropriately applied to the patient.
